(19) United States
(12) Patent Application Publication
(10) Pub. No.: US 2019/0117117 A1
(43) Pub. Date: Apr. 25, 2019

(54) SENSOR, SYSTEM, AND METHOD FOR MONITORING LUNG INTEGRITY

(71) Applicants: Daniel DEARMOND, San Antonio, TX (US); Nitin DAS, San Antonio, TX (US)

(72) Inventors: Daniel DEARMOND, San Antonio, TX (US); Nitin DAS, San Antonio, TX (US)

(73) Assignee: The Board of Regents of the University of Texas System, Austin, TX (US)

(21) Appl. No.: 16/091,366

(22) PCT Filed: Apr. 4, 2017
(86) PCT No.: PCT/US2017/025957

§ 371 Date: Oct. 4, 2018

Related U.S. Application Data
(60) Provisional application No. 62/317,857, filed on Apr. 4, 2016.

(51) Int. Cl. A61B 5/08; A61B 5/00; A61M 1/00; A61B 5/053

(52) U.S. Cl. CPC A61B 5/0803; A61B 5/6865; A61B 5/0205; A61B 5/0538; A61B 5/6858; A61M 1/008

(57) ABSTRACT

The present invention relates to systems and methods for measuring and monitoring physiological changes in a body. More particularly, the invention relates to systems and methods for measuring and monitoring the environment in the vicinity of the lung.

### PRIORITY PARAGRAPH

This Application claims priority to U.S. Provisional Patent Application Ser. No. 62/317,857 filed Apr. 4, 2016, which is incorporated herein by reference in its entirety.

### BACKGROUND

Surgery involving organs in the internal compartments of the chest (e.g., lung, heart), blunt or penetrating trauma involving the chest, or other non-surgical and non-traumatic conditions affecting the lungs may result in anatomic and physiologic changes to the lung and the space around the lungs (pleural space) that may substantially impair the function of the lungs leading to patient morbidity and mortality. Chest tubes, i.e., drainage catheters that are placed in the pleural space in contact with the heart or lung, are commonly used in these clinical scenarios. Changes affecting the internal organs and anatomy of the chest may occur rapidly leading to rapid pathologic changes, and real-time monitors of the internal organs of the chest are in current clinical use to help minimize morbidity and mortality. However, real-time, continuous monitoring of the lung in general is carried out only indirectly by monitoring of non-specific vital signs, or when direct monitoring of the lung is performed it is not continuous or in real-time, for example when radiologic examination of the lungs is performed. There is a clear clinical need for a real-time, continuous monitor of lung/pleural pathology in which direct measurements from the lung/pleural space are performed.

### SUMMARY

The present invention relates to systems and methods for measuring and monitoring physiological changes in a body. More particularly, the invention relates to systems and methods for measuring and monitoring the environment in the vicinity of or across the surface of the lung. In certain aspects the devices, systems, and methods described herein can be used to detect the presence of a pneumothorax, pleural effusion, or pulmonary edema through the measurement of signals produced by devices, methods, and systems described herein. The signals can be generated by monitoring electrical resistance and impedance directly in the pleural space and/or from the surface of the lung via electrode(s) and/or sensor(s) located in the pleural space. As used herein “electrode” refers to a conductor used to establish electrical contact with a nonmetallic part of a circuit, e.g., an organ or the lung surface. In certain aspects electrodes are small metal discs, wires, or looped wire usually made of stainless steel, tin, gold, or silver covered with a silver chloride coating.

Certain embodiments are directed to a lung integrity monitoring system comprising one or more electrodes or sensors operatively coupled to a detector. In certain aspects the detector is voltmeter. Certain embodiments are directed to detecting pneumothorax, hydrothorax, or pleural effusion by monitoring the impedance between two or more electrodes positioned on the surface of the lung. In certain aspects the electrodes are connected to the same transmission lead that is position across the chest wall and connects the electrode(s) or sensor(s) to a monitor or detector. In other aspects each electrode or sensor is connected to the detector by individual transmission leads. In a further aspect the electrode(s) or sensor(s) are not embedded in a permeable matrix. In still a further aspect the electrodes are in direct contact with the lung surface. The detector is configured to monitor changes in impedance as well as compare real-time impedance patterns with reference impedance patterns. The reference impedance pattern(s) can be a baseline reading from the subject being monitored or a reference pattern from another normal and/or abnormal subject(s). A baseline impedance pattern indicates a normal lung condition where as an impedance pattern that is altered or abnormal indicates the presence or formation of an abnormal condition, such as a pneumothorax, hydrothorax, or pleural effusion. In certain aspects the detection of such an abnormal impedance pattern will trigger an alarm or alert. In certain aspect the alarm or alert is sent to medical personnel via an electronic communication such as a text message or the like. In certain aspects the alarm or alert can be an audible alarm or alter that can be heard by medical personnel that are locally situated (e.g., in the same room or proximity) or remote (e.g., at a monitoring station or the like).

Certain embodiments are directed to a thoractostomy tube for monitoring lung integrity comprising a thoractostomy tube having a proximal and distal end, the distal end configured for insertion into the chest of a subject and comprising one or more sensors, the sensors having a sensor head comprising two or more wires forming arcs or electrodes in the thoractostomy tube wall having a non-embedded portion on the surface of the thoractostomy tube, the wires or electrodes configured to contact the lung surface, intrathoracic milieu, or a surface of a non-lung intrathoracic structure or organ. The thoractostomy tube can further comprise at least one articulation. The articulation can be configured to improve the signal-to-noise ratio of the signal generated by the sensors so as to maximize the detection of lung pleura to chest wall pleura contact, that is pleura-to-pleura apposition. The at least one articulation member is configured to be actuatable to cause a change in a bend angle or arc of the thoractostomy tube so as to articulate the distal portion of the tube. The articulation can be 3 to 30 cm from the distal end of the thoractostomy tube. The articulation is configured to position the outer surface of a wire or electrode. In certain aspects the articulation can be used to contact a intrathoracic surface with the electrodes of the thoractostomy tube. In certain aspects one or more sensors are position in the distal 2 to 20 cm of the thoractostomy tube. The non-embedded portion of the electrode or wire can be about 0.1 to 5 mm in length. The non-embedded portion of the electrode or wires can have a diameter of about 0.01 to 0.5 mm. In certain aspects the non-embedded portion of the electrode or wires is copper, stainless steel, or titanium. The wire can have an arc having a radius of curvature of about 0.5 to 4 mm. In further aspects wires diverging from each other once they leave a transmission lead until the apex of the arc where the wires then converge and are coupled to the protective cap. The electrodes or wires can have a minimal spacing of at least 0.1 mm and a maximum spacing up to 2 cm. The sensors can be coupled to a transmission lead that is coupled to a detector.

One embodiment can be directed to a method for detecting pulmonary edema of a subject comprising contacting a lung surface with two or more electrodes that are connected to a detector, and monitoring the impedance over time forming an impedance pattern, wherein deviation from baseline or a reference impedance pattern indicates the presence of pulmonary edema of the subject.

Another embodiment can be directed to a method for detecting lung ventilation of a subject comprising contacting a lung surface with two or more electrodes that are connected to a detector, and monitoring the impedance over time forming an impedance pattern, wherein deviation from baseline or a reference impedance pattern indicates hyperventilation or hypoventilation of the subject.

In certain embodiments two or more electrodes, a sensor, or multiple sensors can be placed in the intrapleural space adjacent to and in contact with the lung. In certain aspects the electrode(s) or sensor(s) are configured to have a sensor face and a support. The sensor face is configured so that the electrode(s) or sensor(s) contact the lung surface. The support surface is configured to contact the inner chest wall. In certain aspects the support surface is non-conductive and insulates the electrode(s) or sensor(s) from the chest wall. In certain aspect the support surface is a polymer. In a further aspect the support surface can be a portion of a chest tube or other device being utilized in the pleural space. Thus, electrical changes that are detected can be attributed to the lung. In certain aspects the electrodes can be positioned on the surface of the lung. The electrodes can be positioned such that the distance between any two electrodes is at least, at most, or about 0.1, 1, 5, 10, 15, 20, 25, 50, 75, 100, 125, 150, 175, to 200 mm, or any values or range there between. In a further aspect the electrode(s) or sensor(s) can be specifically designed with a geometric structure intended to increase sensor or sensors contact with the pleural surface of the lung. Each electrode can be connected by a transmission lead that traverses the chest wall. Each lead can be position in the same or different hole in the chest.

Certain embodiments are directed to a sensor for monitoring lung integrity comprising a sensor head having a proximal end operatively coupled to a transmission lead and a distal end coupled to a protective cap, the sensor head comprising at least four bare or non-embedded sensor wires forming arcs from the transmission lead to the protective cap, the sensor wires each being coupled to the protective cap and forming a concave shape that is configured to contact the curvature of the lung surface and a convex shape that is configured to face the inner chest wall of a subject. In certain aspects the sensor head is attached to a support surface. The support surface can be a non-conducting polymer. In certain aspects the support surface is configured to present the sensor wires to the surface of the lung. The support can have a concave shape. The sensor wire, that is the non-embedded portion of the wire, can be about 0.1, 0.2, 0.5, to 0.6, 1.0, 1.5, 2 mm in length. The sensor wire can have a diameter of about 0.01, 0.2, 0.3 to 0.3, 0.4, 0.5 mm. In certain aspects the sensor wire is made of a conductive metal or metal alloy. In certain aspects the sensor wire is copper or a copper alloy. The sensor wire can be curved and have a radius of curvature of about 0.5 to 4 mm. In certain aspects sensor wires diverge from each other once leaving the transmission lead until the apex of the arc where the wires then converge and are coupled to the protective cap. In a further aspect sensor wires have a minimal spacing of at least 0.1 mm and a maximum spacing up to 2 mm. In certain embodiments the sensor wires are parallel to each other once they leave the transmission lead and are coupled to the protective cap. The transmission lead is configured to couple the sensor head to a detector. In certain aspects the detector comprises a voltmeter.

Certain embodiments are directed to a method for monitoring lung integrity in a subject comprising inserting an intrapleural sensor comprising a sensor head into the pleural space of a subject wherein the sensor head contacts the exterior surface of the lung, the sensor head having a proximal end operatively coupled to a transmission lead and a distal end coupled to a protective cap, the sensor head comprising at least four non-embedded wires forming arcs from the transmission lead to the protective cap forming a concave shape that is configured to contact the lung surface and a convex shape that is configured to face the inner chest wall of a subject. In certain aspects the bare or non-embedded portion of the sensor wires is about 0.1 to 2 mm in length. In a further aspect the bare or non-embedded portion of the sensor wires has a diameter of about 0.01 to 0.5 mm. The bare or non-embedded portion of the sensor wires is made of a metal or metal alloy, e.g., copper, stainless steel, or titanium. In certain aspects the sensor wire has a radius of curvature of about 0.5 to 4 mm. In one aspect the sensor wires diverge from each other once they leave the transmission lead until the apex of the arc where the wires then converge and are coupled to the protective cap. The sensor wires can have a minimal spacing of at least 0.1 mm and a maximum spacing up to 2 mm. In other aspects the sensor wires are parallel to each other once they leave the transmission lead and are coupled to the protective cap.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1A-1B (A) illustration of a top view of one embodiment of a sensor head. (B) illustration of a side view of one embodiment of a sensor head.

FIG. 2 illustrates an experimental set up for demonstration of the functionality of the pleural sensor.

FIG. 3 illustrates an electrical resistance waveform generated in an experimental animal.

FIG. 4 illustrates a graph of resistance change with varying tidal volumes as delivered by the small animal ventilator for three experimental animals.

FIG. 5 shows electrical resistance waveforms generated in an experimental animal while the animal was completely mechanically ventilated versus breathing spontaneously.

FIG. 6 shows an experimental set up for demonstration of the functionality of the pleural sensor as a detector of pneumothorax or pleural effusion (hydrothorax).

FIG. 7 shows an example of the electrical resistance waveform observed during creation of a pneumothorax in the experimental animal.

FIG. 8 represents a dose-response curve showing the resistance change for volume of pneumothorax versus volume of pneumothorax.

FIG. 9A-9B show an example of electrical resistance waveforms observed during creation and resolution of a pleural effusion (hydrothorax) in the experimental animal.

FIG. 10A-10B show an example of electrical resistance waveforms observed during the development of pulmonary edema in an experimental animal.

FIG. 11 illustrates an embodiment of the sensor comprising a support.

FIG. 12 illustrates an embodiment where the sensors are embedded in a thoracostomy tube.

FIG. 13 illustrates an embodiment that is an articulating thoracostomy tube.

FIG. 14 shows experimental results obtained from a prototype of the system described in FIG. 13 when the thoracostomy tube is in the presence of an air leak in the pleural space.

### DESCRIPTION

Organs, tissue, and fluid in a human body possess an ability to conduct electricity due to their components and environment. A body is composed of cells, fluid, and/or air compartments that include a mixture of electrically conductive and resistive components. The mixture of conductive and resistive components determines the electrical properties of a given environment or portion of a body. Impedance, as used herein, refers to the ability to resist electrical currents. Cells, fluid, and/or air spaces all contribute to the electrical impedance that a given location, environment, organ, or tissue exhibits. When physiological changes occur at a particular location in the body the electrical impedance at that location may be measurably altered. The presence of fluid and/or air in a location may be detected by a change in impedance. A monitoring system comprising one or more sensors positioned in a body can allow precise, second by second monitoring of changes in a body as manifested by impedance changes. In certain aspects such a monitoring system can provide precise, second by second monitoring of pneumothorax, pleural effusion, or pulmonary edema as detected by changes in impedance.

Pleural, pulmonary or cardiac pathology has the potential to be rapidly detrimental to the health of patients if not fatal by affecting the function of the lungs and/or pleural space. No real-time continuous monitoring system that takes readings directly from the surface of the lung or pleural space is currently available. Chest tubes are commonly in use in pleural, pulmonary, or cardiac pathology and come into direct contact with the lung, pleural space, or heart, but these tubes are not being used as sensors or to gather data; alternatively, sensors placed within the pleural space could function independently of chest tubes. Electrical impedance/resistance measurement may be the preferred method of sensitively identifying pathologic changes to the lung/pleural space due to the inherent electrical resistance of the air-filled lung. The current device place electrical impedance/resistance measuring sensors into the internal compartments of the chest, either free-standing or incorporated into a chest tube, to detect pneumothorax, pleural effusion, or pulmonary edema with high sensitivity. The current device describes modifications to previously described electrode configurations to maximize the electrical signal from the lung.

A pneumothorax is an abnormal collection of air or gas in the pleural space that causes an uncoupling of the lung from the chest wall. Like pleural effusion (liquid buildup in the pleural space), pneumothorax may interfere with normal breathing. A primary pneumothorax is one that occurs spontaneously without an apparent cause and in the absence of significant lung disease, while a secondary pneumothorax occurs in the presence of existing lung pathology. A pneumothorax can be caused by physical trauma to the chest, or as a complication of medical or surgical intervention. In some cases the amount of air in the chest increases markedly when a one-way valve is formed by an area of damaged tissue, leading to a tension pneumothorax. This condition is a medical emergency that can cause steadily worsening oxygen shortage and low blood pressure. Unless detected and reversed by effective treatment, these sequelae can progress and cause death. Symptoms typically include chest pain and shortness of breath. Diagnosis of a pneumothorax by physical examination alone can be difficult or inconclusive, so a chest radiograph or computed tomography (CT) scan is usually used to confirm its presence.

Because the lungs are an inherently electrically resistive organ owing to the presence of air within the pulmonary alveoli (lung air sacs), the measurement of electrical resistance/impedance from the lung has been suggested as a sensitive detector of lung pathology and devices which measure electrical resistance/impedance from the lung are presently commercially available. However, these existing devices and methods rely on electrodes placed on the skin of the chest wall, external to the organs and anatomic locations of interest (e.g., lung, pleural space) and therefore may be limited in sensitivity and/or specificity in terms of diagnosing pathologic states inside the chest. This represents a missed clinical opportunity because sensors placed on chest tubes, which are commonly placed in situations of internal chest pathology, could be used to convey vital information about the state of the lung, pleural space, heart, or other intrathoracic organs.

In one embodiment, a method of monitoring physiological changes in a body includes: inserting one or more sensors in a body opening (e.g., via puncturing the chest wall) and positioning the sensor in the pleural space contacting the lung—the sensors being operatively connected to a detector. Once the sensor(s) is in place the electrical properties of the one or more sensors appropriately located can be monitored. In certain aspects to sensor and the monitoring system is adapted to measure or detect pneumothorax, pleural effusion (hydrothorax), and pulmonary edema in a subject.

The sensors described herein may be inserted in the pleural space (i.e., pleural sensors) and be coupled to a detector by a transmission lead. The transmission lead can connect one or more sensors to a measurement determining unit or detector. Sensors may transmit data to a detector. A detector can be configured to produce a signal when the impedance/resistance deviates beyond a pre-selected range or to monitor the impedance/resistance over time, thus detecting alterations in the environment surrounding a sensor. The detector may be any device capable of analyzing data from a sensor. In certain aspects the detector can be an impedance monitoring unit. In a further aspect the detector can comprise a voltmeter. In certain aspects the transmission lead extending from the sensor can be arrange such that the transmission lead exits a body or body cavity via a retractor conduit. The transmission lead can be configured to resist damage from fluids in a body cavity. The transmission lead can be insulated or embedded in a protective coating. In certain embodiments the transmission lead can be attached to or embedded in a chest tube or other device with the bare or non-embedded wires projecting from or exposed on the surface of the device to form a sensor head as described herein.

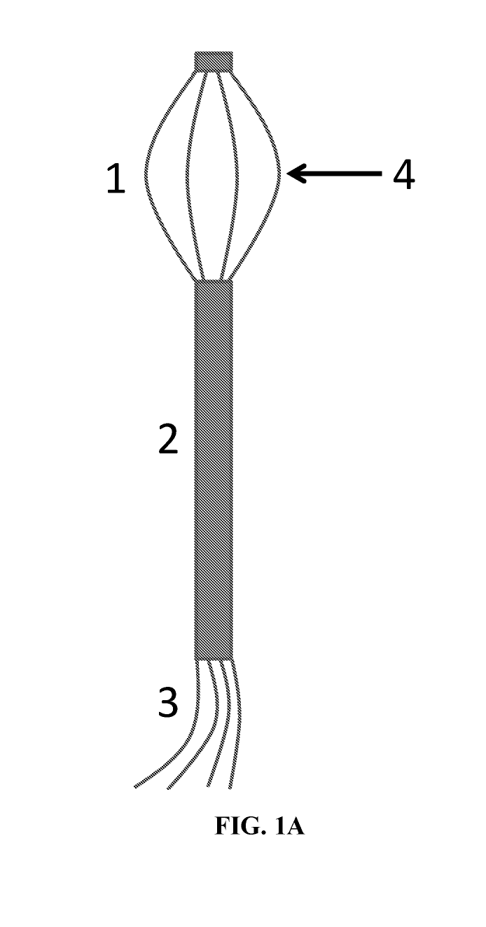

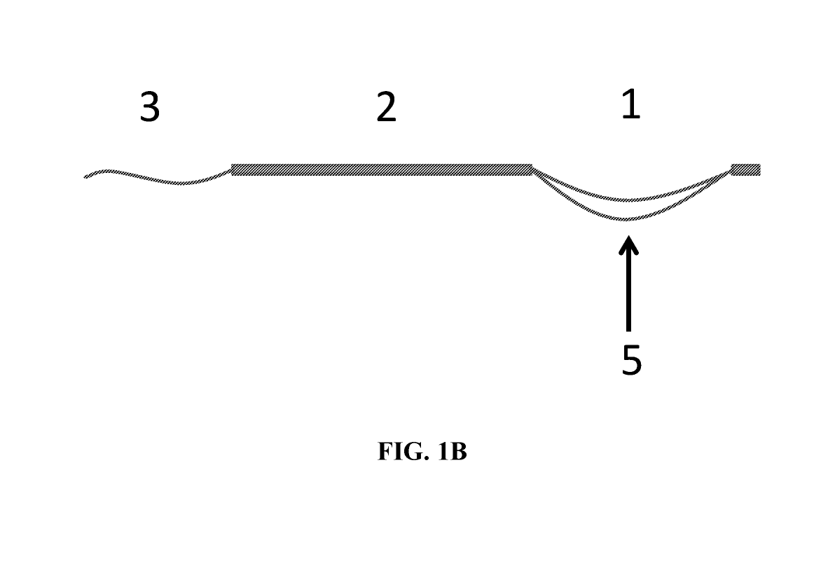

In one embodiment, the pleural sensor would consist of one or more bare metal wires configured in such a way as to promote contact of the wires with the surface of the lung or with the surface of the chest wall, diaphragm, or pericardium. FIGS. 1A and 1B show such a 4-wire version of the sensors in top view (FIG. 1A) and side view (FIG. 1B). In certain aspects the sensor consists of an exposed portion of a 4-wire array intended to be placed within the pleural space (1) a portion of the wires which is insulated which extends out of the thorax (2) forming a transmission lead, and a second portion of exposed wire (3) forming the sensor head intended to be connected to the resistance measuring device. In certain aspects the portion of exposed wires (1) intended to be placed within the pleural space would be configured to create a central expansion of the wires as seen from the top view (4), and a convex shape as seen from the side view (5), to promote contact of the wires with the organ targeted for electrical measurements, e.g. the lung, chest wall, diaphragm, or pericardium.

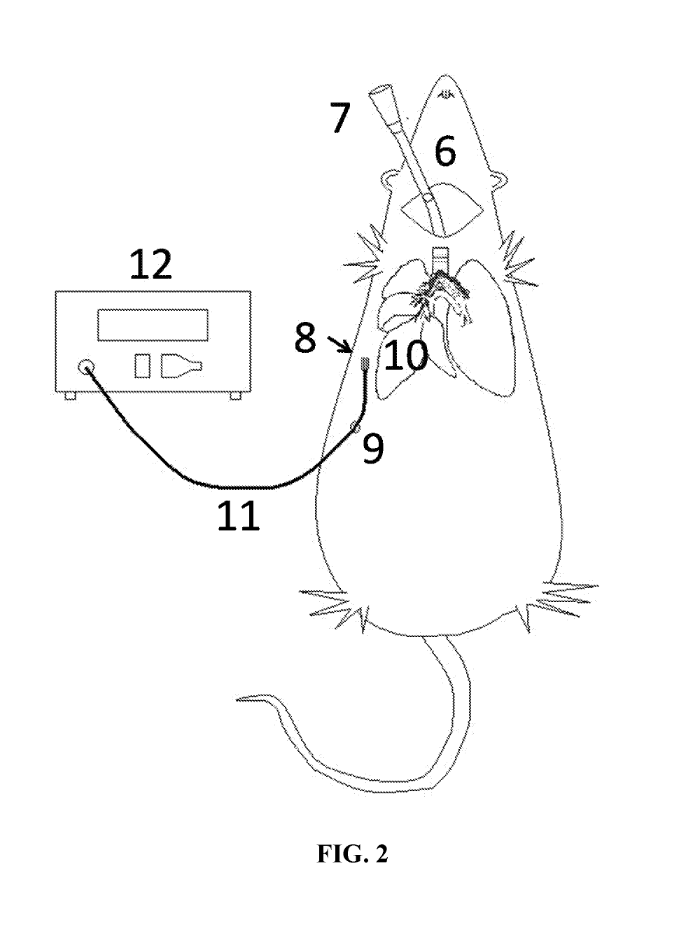

FIG. 2 illustrates an experimental set up for demonstration of the functionality of the pleural sensor. A Sprague-Dawley rat (6) was maintained under general anesthesia on a small animal ventilator (not shown) via a tracheostomy (7). The sensor array (8) as described herein was inserted into the pleural space via a stab incision in the chest wall (9) so that it could be brought into contact with the lung (10) using the convexity of the wires to promote contact with the lung and avoid contact with the chest wall (parietal pleura). An insulated section of wires (11) (transmission lead) was brought out through the stab incision (9) and connected to the resistance measuring device or detector (12).

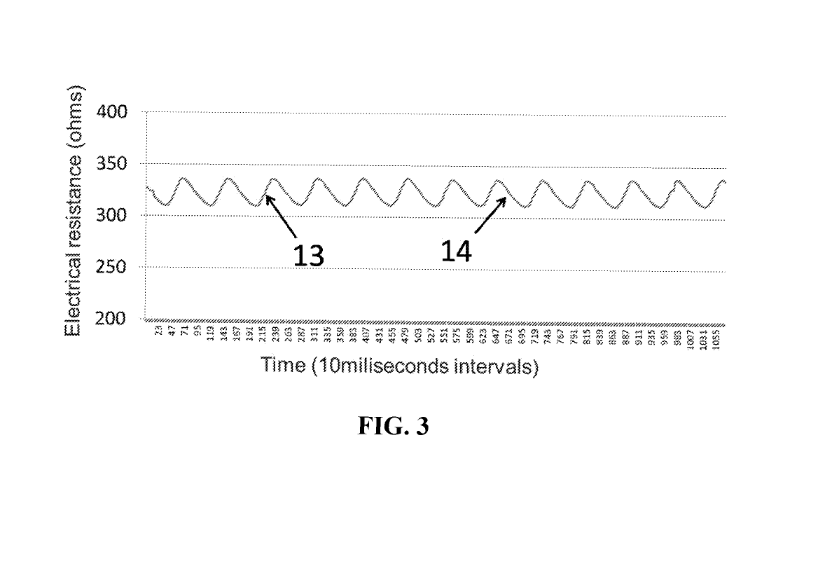

FIG. 3 shows an electrical resistance waveform generated in the experimental animal of FIG. 2. Video recording in real time (not shown) of the resistance waveform in the same frame as the piston of the small animal ventilator delivering breaths to the animal was performed to correlate components of the resistance waveform to phases of the respiratory cycle. This video recording demonstrated that upstrokes in the resistance waveform (13) caused by an increase in electrical resistance as detected by the sensors were associated with the inspiratory phase of the respiratory cycle and downstrokes in the resistance waveform (14) caused by a decrease in the electrical resistance measured by the sensors were associated with the expiratory phase of the respiratory cycle.

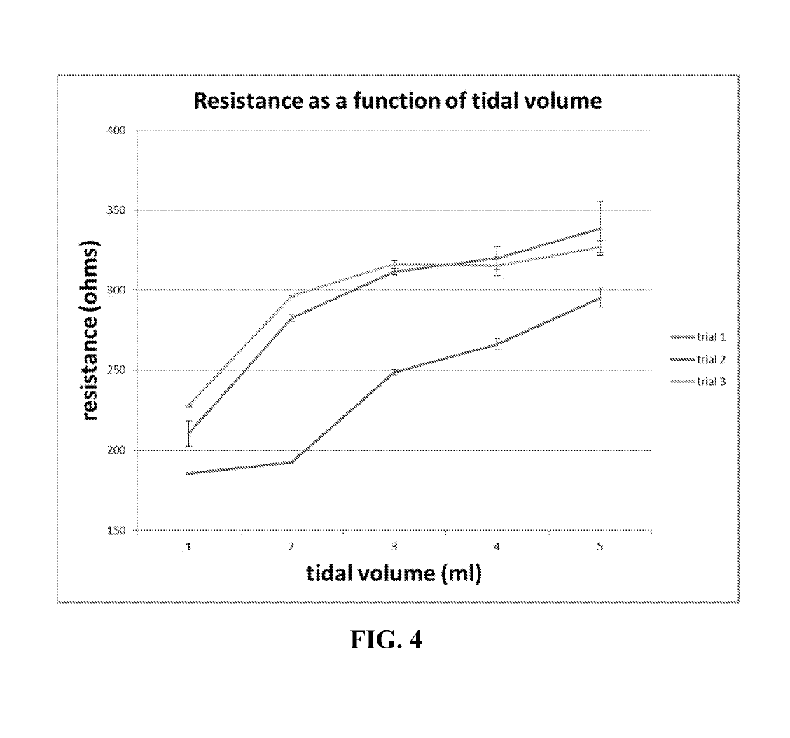

FIG. 4 shows a graph of resistance change with varying tidal volumes as delivered by the small animal ventilator for three experimental animals following the experimental set-up illustrated in FIG. 2. Three Sprague-Dawley rats of approximately 300 gm weight were placed on the small animal ventilator with the electrical resistance sensor contacting the lung of the experimental animal as shown in FIG. 2 and the tidal volume of the breaths delivered to the animal by the small animal ventilator was modulated to determine the effect on the resistance measured by the sensor. Tidal volumes of 1 ml, 2 ml, 3 ml, 4 ml and 5 ml were delivered to the experimental animal; for animals of this weight, 3 ml tidal volume represented normo-ventilation, 1 ml tidal volume represented hypoventilation, and 5 ml represented hyperventilation. A dose-dependent increase in electrical resistance measurements from the lung was observed with increasing tidal volumes delivered to the animal. This demonstrated the ability of the sensor to detect and differentiate between hypo-, normo-, or hyperventilation of the lung.

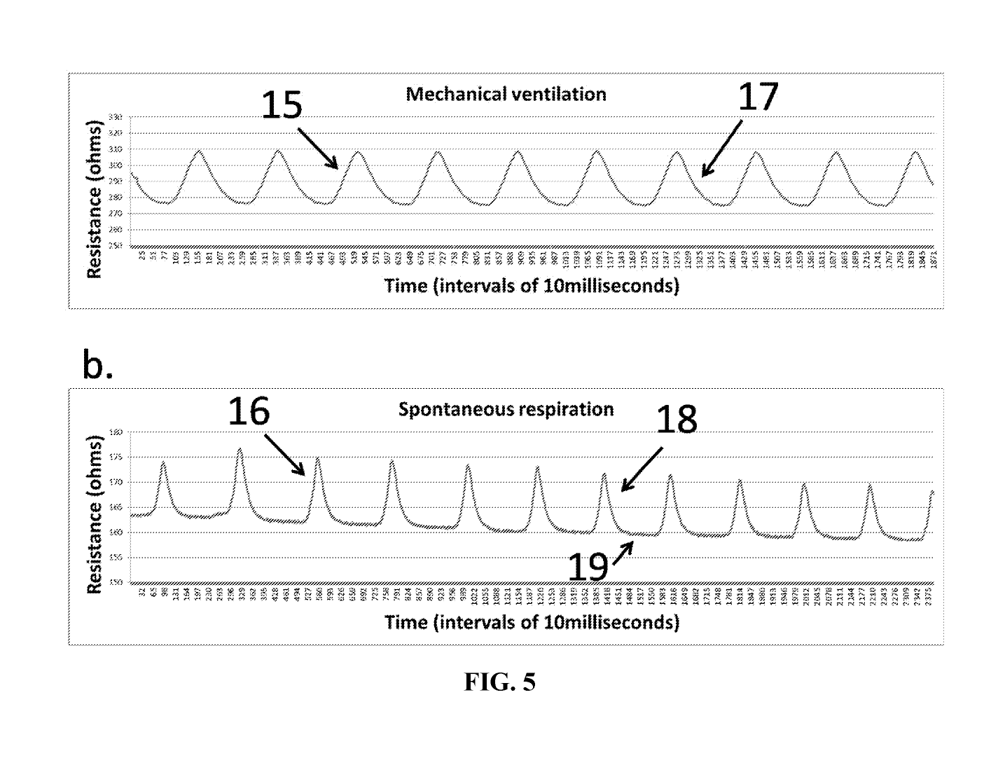

FIG. 5 shows electrical resistance waveforms generated in the experimental animal of FIG. 2 while the animal was completely mechanically ventilated versus breathing spontaneously. In FIG. 2A, the experimental animal was under deep general anesthesia resulting in complete suppression of spontaneous, animal-initiated breathing and resulting in all observed breaths being delivered by the small animal ventilator. In FIG. 2B, the general anesthetic amount delivered to the experimental animal was decreased to maintain general anesthesia but allow for the return of spontaneous respirations; in this case, the experimental animal was removed from the small animal ventilator and allowed to inhale anesthetic and oxygen via a small animal nose-cone via spontaneous, animal-initiated respirations. This experiment showed the sensitivity of the resistance-detecting sensors to differentiate between the respiratory patterns of mechanical ventilation and spontaneous breathing: the upstroke in the resistance curve during the inspiratory phase of mechanical ventilation (15) was more gradual than the sharp upstroke in the resistance curve during the inspiratory phase of spontaneous breathing (16); the downstroke in the resistance curve associated with the expiratory phase of mechanical ventilation (17) was fairly uniform in slope while the downstroke in the resistance curve associated with the expiratory phase of spontaneous breathing was marked by a steep early decrease (18) followed by a more prolonged, flattened phase (19) during which resistance continued to decrease more slowly. Additionally, in the mechanically ventilated rat, the ratio of the time duration of the inspiratory phase (15) to the time duration of the expiratory phase (17) was approximately 1:1.5, while in the spontaneously breathing rat, the ratio of the time duration of the inspiratory phase (16) to the time duration of the expiratory phase (18+19) was approximately 1:4. This demonstrated the ability of the sensor to detect and distinguish the inspiratory to expiratory ratio (I:E ratio), an important parameter in respiratory medicine, in differentially ventilated animals.

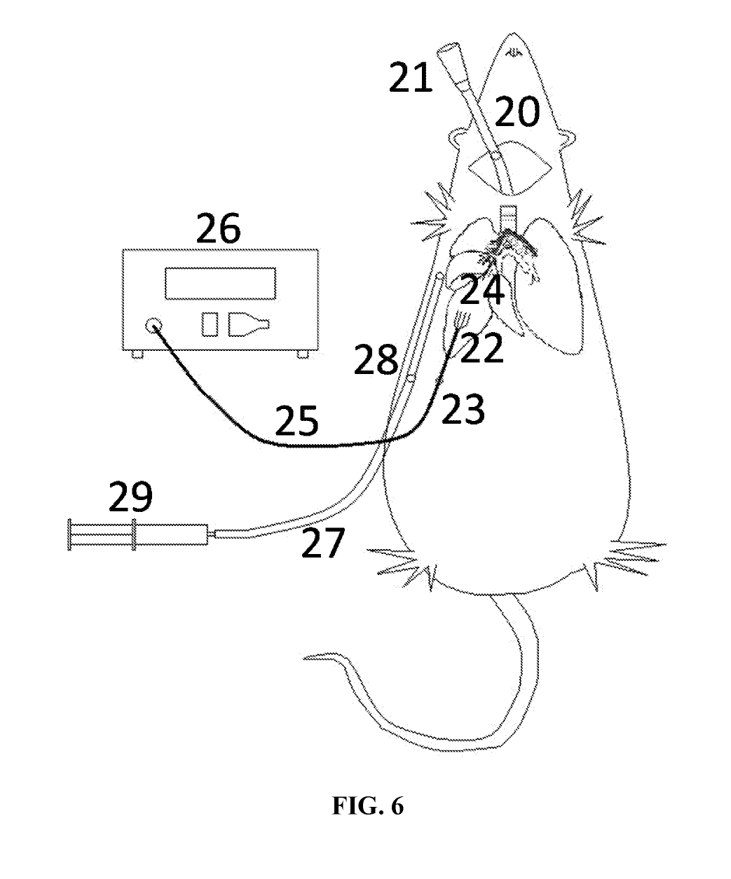

FIG. 6 shows an experimental set up for demonstration of the functionality of the pleural sensor as a detector of pneumothorax or pleural effusion (hydrothorax). A Sprague-Dawley rat (20) was maintained under general anesthesia on a small animal ventilator (not shown) via a tracheostomy (21). The four-wire sensor array (22) as described in FIG. 1 was inserted into the pleural space via a stab incision in the chest wall (23) so that it could be brought into contact with the lung (24) using the convexity of the wires to promote contact with the lung (24) and avoid contact with the chest wall (parietal pleura). An insulated section of wires (25) (transmission lead) was brought out through the stab incision (23) and connected to the resistance measuring device or detector (26). Additionally, a 14 gauge flexible tube (27) was placed into the pleural space by a separate stab incision (28) and attached to a syringe (29). The syringe (29) was used to insufflate varying volumes of air into the pleural space via the flexible tube (27) to create varying degrees of pneumothorax or varying volumes of 0.9% NaCl to create varying degrees of pleural effusion (hydrothorax).

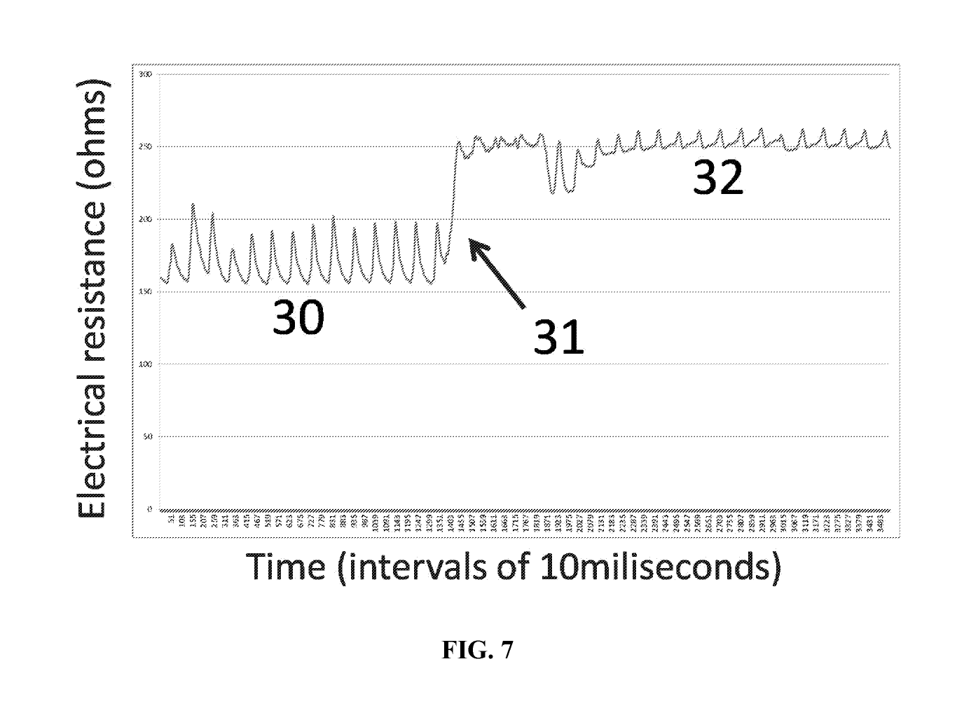

FIG. 7 shows an example of the electrical resistance waveform observed during creation of a pneumothorax in the experimental animal depicted in FIG. 6. First, a baseline electrical resistance from the surface of the lung was obtained (30). Next, pneumothorax was created by injecting air into the pleural space during which a change in electrical resistance was observed (31). Finally, a new baseline electrical resistance was observed representing a new steady-state resistance waveform for the pleural space in the presence of a stable pneumothorax (32). The average resistance observed from the baseline prior to pneumothorax creation (30) was subtracted from the average resistance from the new, post-pneumothorax resistance waveform (32) to determine the change in resistance due to creation of the pneumothorax. Alternatively, area under the curve for the baseline resistance curve prior to pneumothorax creation (30) could be subtracted from the area under the curve for the new, post-pneumothorax resistance waveform (32) to determine the change in resistance due to creation of the pneumothorax.

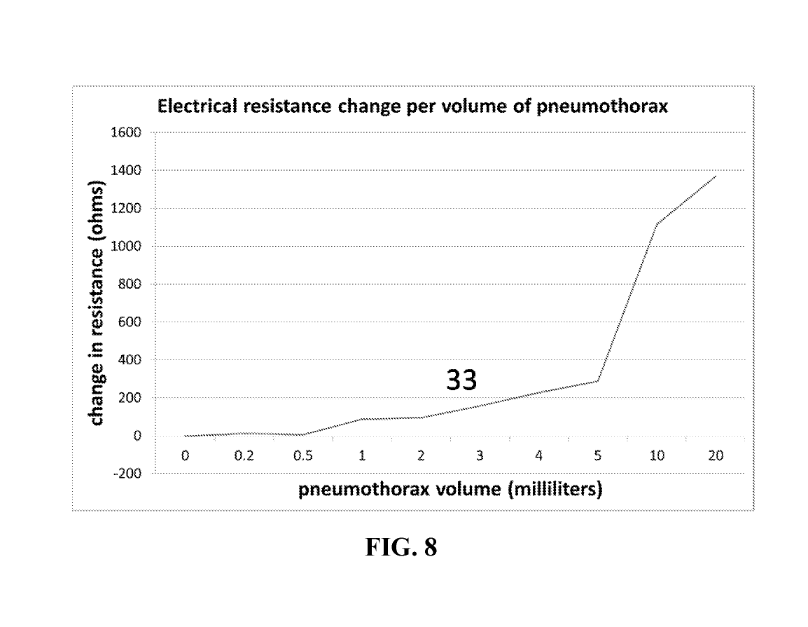

FIG. 8 represents a dose-response curve showing the resistance change for volume of pneumothorax versus volume of pneumothorax. All measurements shown in FIG. 8 were performed in one animal according to the schema presented in FIG. 6. Pneumothorax was created using the following volumes of air insufflated into the pleural space: 0.2 ml, 0.5 ml, 1 ml, 2 ml, 3 ml, 4 ml, 5 ml, 10 ml, 20 ml. After each volume of pneumothorax was induced and electrical measurements carried out, the pneumothorax was aspirated by drawing back on the syringe (FIG. 6, 29) until the original resistance baseline (FIG. 7, 30) was restored. This allowed one experimental animal to undergo repeated pneumothorax at varying volumes. Electrical resistance average values from before and after induction of pneumothorax were compared to determine the change in resistance for the varying magnitudes of pneumothorax. A dose-dependent relationship between increasing pneumothorax volume and increasing positive change in electrical resistance was observed (33). This showed the ability of the sensor to detect and differentiate varying volumes of pneumothorax and to detect the resolution of a pneumothorax by air evacuation from the pleural space by restoration of the original resistance baseline.

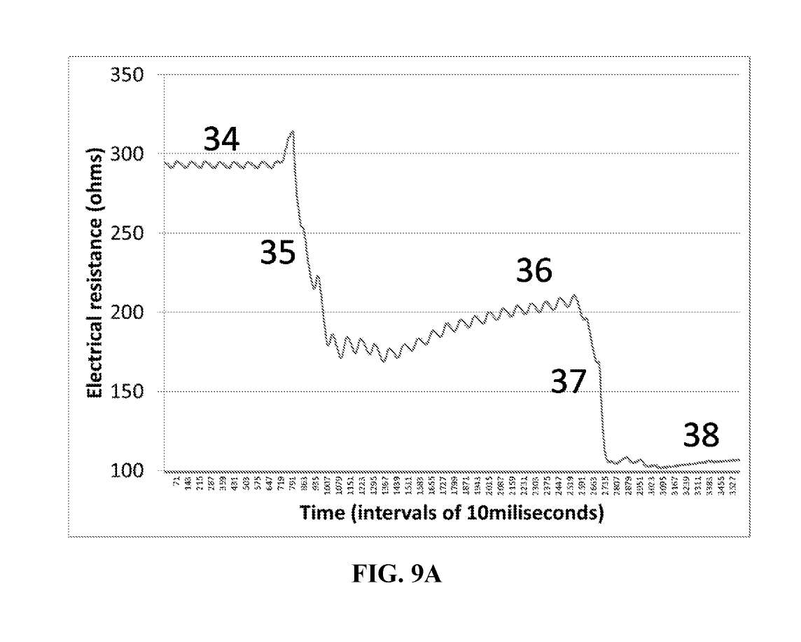

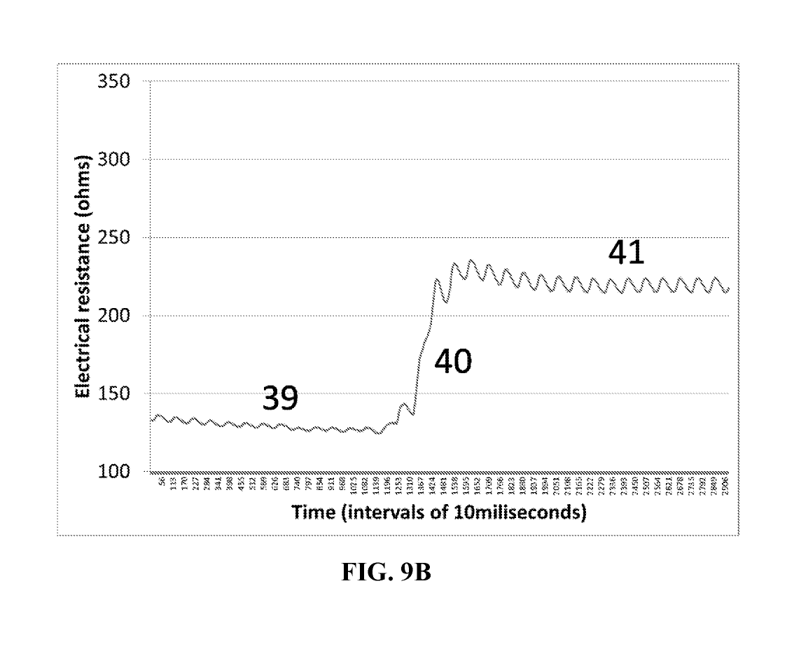

FIGS. 9A and 9B shows an example of electrical resistance waveforms observed during creation and resolution of a pleural effusion (hydrothorax) in the experimental animal depicted in FIG. 6. First, a baseline electrical resistance from the surface of the lung was obtained (FIG. 9A, 34). Next, pleural effusion was created by injecting 5 cc 0.9% NaCl into the pleural space during which a change (decrease) in electrical resistance was observed (FIG. 9A, 35). Finally, a new baseline electrical resistance was observed representing a new steady-state resistance waveform for the pleural space in the presence of a stable pleural effusion (hydrothorax) (FIG. 9A, 36). An additional 2 cc 0.9% NaCl was injected into the pleural space during which an additional change (decrease) in electrical resistance was observed (FIG. 9A, 37) leading to an additional new baseline resistance waveform representing a new, lower, steady-state resistance waveform for the pleural space in the presence of a larger, stable pleural effusion (hydrothorax) (FIG. 9A, 38). In FIG. 9B, electrical resistance waveforms are shown representing the electrical properties of the pleural space during aspiration of the pleural effusion created in FIG. 9A. First, the steady state electrical resistance baseline waveform of the pleural space with the large effusion created in FIG. 9A was measured (39). Next, the syringe (FIG. 6, 29) attached to the tube entering the pleural space (FIG. 6, 29) was used to aspirate and evacuate the pleural effusion created in FIG. 9A, leading to a change (increase) in the electrical resistance waveform (40); this ultimately led to the establishment of a new steady-state resistance waveform (41) representing partial resolution of the pleural effusion. The experimental results shown in FIGS. 9A and 9B illustrate the ability of the sensor to detect the development and resolution of pleural effusion (hydrothorax).

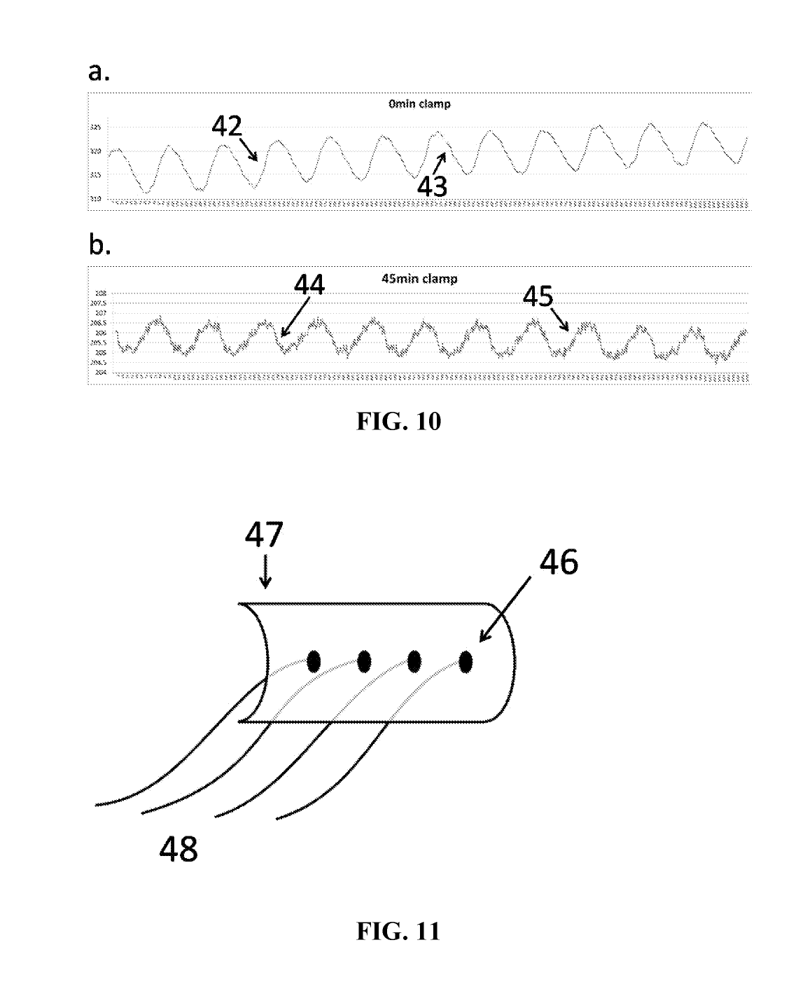

FIGS. 10A and 10B shows an example of electrical resistance waveforms observed during the development of pulmonary edema in an experimental animal. A Sprague-Dawley rat was placed under general anesthesia and the sensor described in FIG. 1 was placed in contact with the lung according to the schema describe in FIG. 2. A surgical clip was then placed on the pulmonary vein of the lung (not shown in illustration); this maneuver gradually results in fluid build-up within the lung (pulmonary edema) because pulmonary arterial blood is still pumped by the heart to the lung but with the pulmonary vein occluded the means of egress of pulmonary blood back to the heart is blocked. In FIG. 10A, an electrical resistance waveform was obtained at the 0 minute mark immediately after placement of the pulmonary venous clip; this resistance tracing still appears to reflect an essentially normal lung waveform with upstrokes in the resistance waveform (42) caused by an increase in electrical resistance as detected by the sensors associated with the inspiratory phase of the respiratory cycle and downstrokes in the resistance waveform (43) caused by a decrease in the electrical resistance measured by the sensors associated with the expiratory phase of the respiratory cycle (as shown previously in FIG. 3). Next, in FIG. 10B, an electrical resistance waveform was obtained 45 minutes after placement of the pulmonary venous clip; grossly the lung appeared hemorrhagic and edematous at this time point. The overall resistance waveform was displaced to a lower average resistance and the amplitude of the waveform was diminished when compared to the waveform from FIG. 10A, consistent with a less aerated lung. The resistance tracing from FIG. 10B also showed an inverted waveform with downstrokes in the resistance waveform (44) caused by a decrease in electrical resistance as detected by the sensors associated with the inspiratory phase of the respiratory cycle and upstrokes in the resistance waveform (45) caused by an increase in the electrical resistance measured by the sensors associated with the expiratory phase of the respiratory cycle. Control lungs from the same experimental animal showed a much smaller decrease in average resistance and amplitude and the waveform inversion seen in FIG. 10B was not observed. This experiment showed that the sensor was a very sensitive detector of pulmonary edema and that multiple parameters of the waveform generated by the sensor were altered in pulmonary edema including: average total resistance; amplitude of waveform; and waveform inversion.

In one embodiment of the pleural sensor, rather than have wires bent to favor contact with a target organ or tissue surface, one or more electrodes could be connected or coupled to the surface of a support or a spacer. In certain aspects the support or spacer can be an inert, electrically resistive material to orient the bare or non-embedded wires toward a target organ or tissue surface. FIG. 11 depicts one embodiment of a sensor comprising a spacer. In FIG. 11, four wires (46) consisting of a biocompatible bare metal (such as temporary atrial pacing wires) are built into a half cylinder (47) of some biocompatible insulating material (such as silicone) so as to protrude from the convex surface of the half-cylinder (47) and come into contact with some target tissue that contacts the convex surface but be prevented from contact by a tissue surface disposed towards the concave side of the half cylinder. The sensor wires are connected by a transmission lead (48) to a resistance or impedance measuring device or detector (not shown). In other embodiments the half cylinder could be replace by a thoracostomy tube or other type of surgical drain.

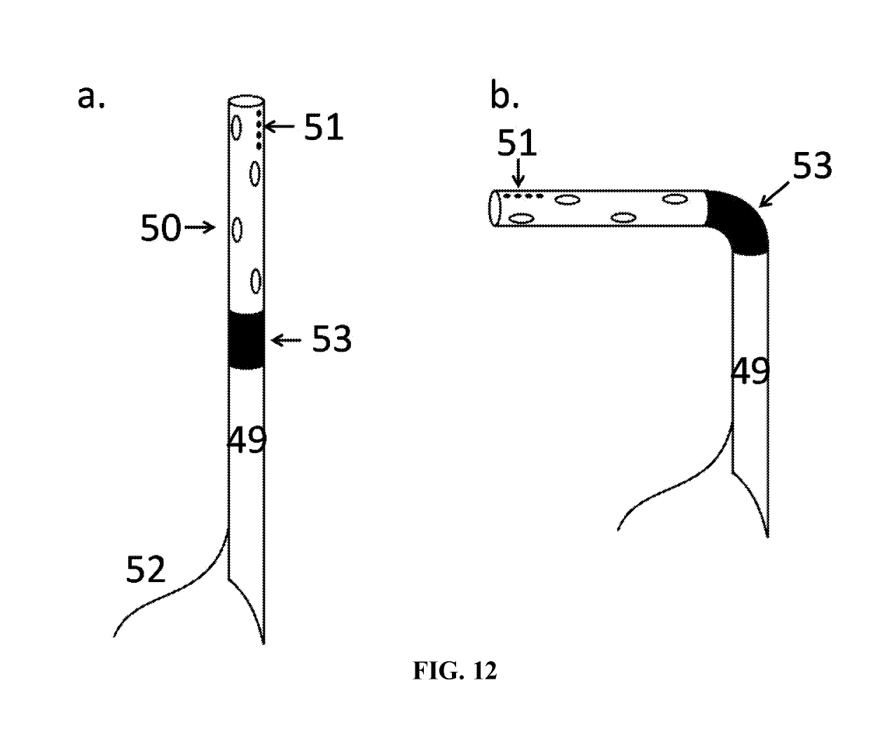

In one embodiment, (FIG. 12a) the sensor or sensors are embedded in a thoracostomy tube (49); such tubes are generally made of soft plastic or silicone material and typically have one or more perforations (50) in the distal end of the tube to facilitate the evacuation of fluid and/or air from the pleural or pericardial space. In FIG. 12a, one or more sensors (51) are embedded in the material of the distal thoracostomy tube so as to be exposed to direct contact with intra-pleural structures or organs; the sensors are connected to wires (52) that traverse the thoracostomy tube embedded in the material of the thoracostomy tube (49) and emerge from the material at the proximal end of the thoracostomy tube (49) where they can be attached to an impedance-measuring device (not shown). In FIG. 12b, an embodiment is depicted in which an articulation point in the thoracostomy tube (53) allows for either permanent or temporary articulation of the thoracostomy tube; this permits more exact orientation of the sensor or sensors (51) within the pleural space so as to optimize detection of the intrapleural impedance signal. Specifically, this articulation feature could be used to orient and maintain the portion of thoracostomy tube (49) containing the sensors (51) either permanently or temporarily in a position immediately adjacent to the chest wall, along the parietal pleura, to improve the signal to noise ratio of the impedance signal from the sensors (51) as a means of more reliably confirming contact between the pleura of the lung with the pleura of the chest wall (i.e., pleura-to-pleura apposition, widely accepted as an important endpoint of thoracostomy tube therapy). Articulation could be effected by a wire/pulley system, as in flexible endoscopes. Additionally, this articulation feature of the thoracostomy tube (49) may improve the functionality its drainage feature by allowing for re-positioning of the tube in the patient to maximize the drainage of air and/or fluid.

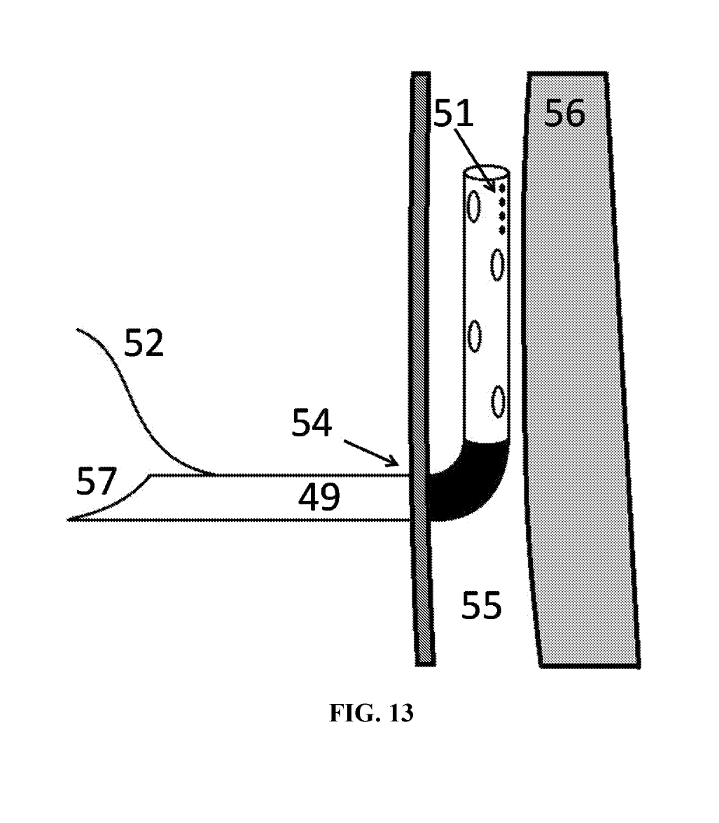

In FIG. 13 the articulating thoracostomy tube (49) described in FIG. 12 is depicted in an articulated state after placement inside the pleural space of a patient in which an air leak is present (not shown) causing air to be introduced into the pleural space. The tube is passed through the chest wall (54) and into the pleural space (55) and made to articulate such that the sensors (50) are disposed towards the lung (56) and away from the chest wall (54) such that contact of the sensors with the lung (56) is promoted while contact of the sensors with the chest wall (54) is prevented. The connecting wires (52) which connect to the sensors (51) are then attached to an impedance detecting device (not shown) outside of the patient. The thoracostomy tube is attached by its most proximal end (57) to a suction system (not shown) which can either apply suction to the tube to evacuate the air leak from the pleural space (55) or apply no suction, allowing air to accumulate in the pleural space (55) as a model of pneumothorax.

A thoractostomy tube described herein can include trigger or other device for controlling the articulation of the distal portion of the tube and the positioning of the wires or electrode. In one example a trigger can be used to apply force against a piston disposed at a proximal end of thoractostomy tube to move the piston in a distal direction. Additionally, a knob can be disposed at a proximal, upper portion of thoractostomy tube on handpiece. The knob can be connected to the articulation segment or portion such that articulation or actuation of the knob provides a corresponding articulation of articulation segment or portion, allowing the thoractostomy tube sensors and the distal end of the tube to be moved to a desired position. Articulation segment or portion may articulate in a plane horizontal, vertical, or horizontal and vertical to the longitudinal axis of thoractostomy tube. A horizontal and/or vertical articulation of knob (e.g., by a user's thumb) effects a corresponding, respective horizontal or vertical articulation of articulation segment or portion. Alternatively, a clockwise or counter-clockwise rotation of knob may effect a corresponding articulation in a selected plane (either the horizontal or vertical plane, for example). The thoractostomy tube may also be rotatable relative to the handpiece about a longitudinal axis defined by the thoractostomy tube.

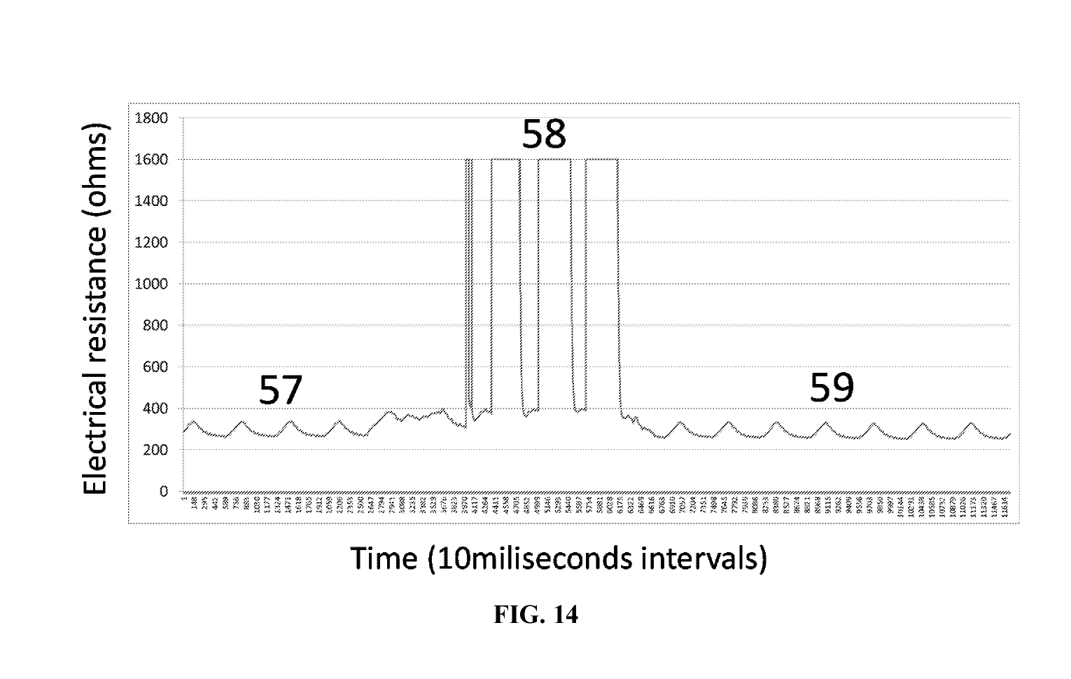

FIG. 14 shows experimental results obtained from a prototype of the system described in FIG. 13 in the presence of an air leak in the pleural space (FIG. 13, 55) of an experimental pig. With suction applied to the thoracostomy tube (FIG. 13, 49) a normal lung waveform is detected by the sensors (FIG. 13, 51) in the electrical resistance tracing (FIG. 14, 57) because the suction is preventing the development of pneumothorax by evacuating air as it accumulates in the pleural space. When suction applied to the thoracostomy tube (FIG. 13, 49) is turned off, air accumulates in the pleural space (FIG. 13, 55), i.e., a pneumothorax develops, causing the lung (FIG. 13, 56) to be separated from the chest wall (FIG. 13, 54); this results in loss of contact of the sensors (FIG. 13, 51) with the lung because the thoracostomy tube in its articulated state is maintained in alignment with the chest wall. In the electrical resistance tracing, this corresponds to a maximization of the electrical resistance measured to the upper limit of resolution of the impedance detecting device (58). This is a clear signal of the loss of visceral pleura-to-parietal pleura contact due to the development of a clinically significant pneumothorax and is possible only with resistance sensors embedded in a thoracostomy tube which is oriented and maintained either permanently or temporarily in a position immediately adjacent to the chest wall, along the parietal pleura as depicted in FIG. 13. Finally, when suction is restored to the thoracostomy tube (FIG. 13, 49), the pneumothorax is evacuated and visceral pleura-to-parietal pleura contact is restored resulting in renewed contact of the lung (FIG. 13, 56) with the sensors (FIG. 13, 51); in the electrical resistance tracing this manifests as a restoration of the normal lung resistance tracing (59). This experiment demonstrated the ability of a prototype of the articulating thoracostomy tube illustrated in FIG. 13 to detect lung collapse by specifically monitoring visceral pleura to parietal pleura apposition and also to detect the effectiveness of therapy to achieve resolution of pneumothorax.

1. A thoractostomy tube for monitoring lung integrity comprising a thoractostomy tube having a proximal and distal end, the distal end configured for insertion into the chest of a subject and comprising one or more sensors, the sensors having a sensor head comprising two or more wires forming arcs or electrodes in the thoractostomy tube wall having a non-embedded portion on the surface of the thoractostomy tube, the wires or electrodes configured to contact the lung surface, intrathoracic milieu, or a surface of a non-lung intrathoracic structure or organ.

2. The thoractostomy tube of claim 1, further comprising an articulation.

3. The thoracostomy tube of claim 2, wherein the articulation is configured to improve the signal-to-noise ratio of the signal generated by the sensors so as to maximize the detection of lung pleura to chest wall pleura contact, that is pleura-to-pleura apposition.

4. The thoractostomy tube of claim 1, wherein the articulation is 3 to 15 cm from the distal end of the thoractostomy tube.

5. The thoractostomy tube of claim 1, wherein one or more sensors are position in the distal 5 to 20 cm of the thoractostomy tube.

6. The thoractostomy tube of claim 1, wherein the non-embedded portion of the electrode or wire is about 0.1 to 5 mm in length.

7. The thoractostomy tube of claim 1, wherein the non-embedded portion of the electrode or wires has a diameter of about 0.01 to 0.5 mm.

8. The thoractostomy tube of claim 1, wherein the non-embedded portion of the electrode or wires is copper, stainless steel, or titanium.

9. The thoractostomy tube of claim 1, wherein the wire arc has a radius of curvature of about 0.5 to 4 mm.

10. The thoractostomy tube of claim 1, wherein the wires diverging from each other once they leave the transmission lead until the apex of the arc where the wires then converge and are coupled to the protective cap.

11. The thoractostomy tube of claim 1, wherein the electrodes or wires have a minimal spacing of at least 0.1 mm and a maximum spacing up to 2 cm.

12. The thoractostomy tube of claim 1, wherein the sensors are coupled to a transmission lead that is coupled to a detector.

13. A method for detecting fluid or air in the pleural space of a subject comprising contacting a lung surface with two or more electrodes that are connected to a detector, and monitoring the impedance over time forming an impedance pattern, wherein deviation from baseline or a reference impedance pattern indicates the presence of fluid or air in the pleural space of the subject.

14. The method of claim 13, wherein the detector is voltmeter.

15. The method of claim 13, wherein the fluid in the pleural space is a pneumothorax, hydrothorax, or pleural effusion.

16. The method of claim 13, wherein the electrode(s) are not embedded in a permeable matrix.

17. The method of claim 13, wherein detection of an abnormal impedance pattern will trigger an alarm or alert.

18. The method of claim 17, wherein the alarm or alert is sent to medical personnel via an electronic communication.

19. The method of claim 13, wherein the electrode(s) are configured to have a sensor face and a support, the sensor face is configured so that the electrode(s) contact the lung surface and the support surface is configured to contact the inner chest wall.

20. The method of claim 19, wherein the support surface is non-conductive and insulates the electrode(s) from the chest wall.

21. The method of claim 13, wherein the electrodes are separated by at least, at most, or about 0.1, 1, 5, 10, 15, 20, 25, 50, 75, 100, 125, 150, 175, to 200 mm of the lung surface.

22. The method of claim 13, wherein the electrode(s) are specifically designed with a geometric structure intended to increase electrode contact with the pleural surface of the lung.

23. A method for detecting pulmonary edema of a subject comprising contacting a lung surface with two or more electrodes that are connected to a detector, and monitoring the impedance over time forming an impedance pattern, wherein deviation from baseline or a reference impedance pattern indicates the presence of pulmonary edema of the subject.

24. A method for detecting lung ventilation of a subject comprising contacting a lung surface with two or more electrodes that are connected to a detector, and monitoring the impedance over time forming an impedance pattern, wherein deviation from baseline or a reference impedance pattern indicates hyperventilation or hypoventilation of the subject.

25. A sensor for monitoring lung integrity comprising a sensor head having a proximal end operatively coupled to a transmission lead and a distal end coupled to a protective cap, the sensor head comprising two or more electrodes or non-embedded wires forming arcs from the transmission lead to the protective cap, the wires each being coupled to the protective cap forming a concave shape that is configured to contact the lung surface and a convex shape that is configured to face the inner chest wall of a subject.

26. The sensor of claim 25, wherein the sensor head is attached to a support surface.

27. The sensor of claim 26, wherein the support surface is a non-conducting polymer.

28. The sensor of claim 25, wherein the non-embedded portion of the wire is about 0.1 to 2 mm in length.

29. The sensor of claim 25, wherein the non-embedded portion of the wires has a diameter of about 0.01 to 0.5 mm.

30. The sensor of claim 25, wherein the non-embedded portion of the wires is copper, stainless steel, or titanium.

31. The sensor of claim 25, wherein the wire arc has a radius of curvature of about 0.5 to 4 mm.

32. The sensor of claim 25, wherein the wires diverging from each other once they leave the transmission lead until the apex of the arc where the wires then converge and are coupled to the protective cap.

33. The sensor of claim 25, wherein the wires have a minimal spacing of at least 0.1 mm and a maximum spacing up to 2 mm.

34. The sensor of claim 25, wherein the wires are parallel to each other once they leave the transmission lead and are coupled to the protective cap.

35. The sensor of claim 25, wherein the transmission lead is configured to couple the sensor head to a detector.

36. A lung integrity monitoring system comprising the sensor of claim 25 operatively coupled to a detector.

37. The system of claim 36, wherein the detector is voltmeter.

38. A method for monitoring lung integrity in a subject comprising inserting a intrapleural sensor comprising a sensor head in to the pleural space of a subject wherein the sensor head contacts the exterior surface of the lung, the sensor head having a proximal end operatively coupled to a transmission lead and a distal end coupled to a protective cap, the sensor head comprising two or more electrodes or non-embedded wires forming arcs from the transmission lead to the protective cap forming a concave shape that is configured to contact the lung surface and a convex shape that is configured to face the inner chest wall of a subject.

39. The method of claim 38, wherein the non-embedded portion of the wires is about 0.1 to 2 mm in length.

40. The method of claim 38, wherein the non-embedded portion of the wires has a diameter of about 0.01 to 0.5 mm.

41. The method of claim 38, wherein the non-embedded portion of the wires is copper, stainless steel, or titanium.

42. The method of claim 38, wherein the wire arc has a radius of curvature of about 0.5 to 4 mm.

43. The method of claim 38, wherein the wires diverging from each other once they leave the transmission lead until the apex of the arc where the wires then converge and are coupled to the protective cap.

44. The method of claim 43, wherein the wires have a minimal spacing of at least 0.1 mm and a maximum spacing up to 2 mm.

45. The method of claim 38, wherein the wires are parallel to each other once they leave the transmission lead and are coupled to the protective cap.
